(19) United States
(12) Patent Application Publication
(10) Pub. No.: US 2024/0075252 A1
(43) Pub. Date: Mar. 7, 2024

(54) Instrument Delivery Device with Linearly Compressible Housing

(71) Applicant: Becton, Dickinson and Company, Franklin Lakes, NJ (US)

(72) Inventors: Adam J. Boud, Bluffdale, UT (US); Shaun Lauer, Portland, OR (US)

(21) Appl. No.: 17/901,034

(22) Filed: Sep. 1, 2022

(51) Int. Cl. A61M 25/06; A61M 25/00

(52) U.S. Cl. CPC A61M 25/0606; A61M 25/0074; A61M 25/0097; A61M 2025/0062; A61M 2025/0079

(57) ABSTRACT

Provided herein is a medical device, including an instrument having a proximal end and a distal end, an introducer configured to moveably receive the instrument and having an outer housing having a proximal end, a distal end, and a sidewall therebetween defining an inner volume and an inner housing having a proximal end, a distal end, and a sidewall therebetween defining an inner volume, the outer housing slidably received within the inner housing, the distal end of the introducer configured to couple the introducer to an intravenous line. The inner and/or outer housing are configured to move relative to each other to move the instrument between a first position and a second position and to reduce an effective length of the device.

### BACKGROUND OF THE INVENTION

### Field of the Invention

The present disclosure relates generally to medical devices for use with intravenous (IV) catheters and, more specifically, to medical devices with features for improving performance in terms of accessing the vasculature.

### Description of Related Art

Instrument delivery devices, including those used for collecting blood, when used with indwelling IV catheters can include displaceable implements that are advanced beyond the tip of the indwelling catheter. Often, when the displaceable implement is advanced, it can encounter an obstruction, resulting in deflection of the implement. Examples of obstructions include the friction of the seal within the delivery device, torturous path within an integrated catheter, pinching of the catheter tubing as it dives into the skin, thrombus, fibrin, and valves. Further, delivery of instruments can be complicated by interchange of instruments, and the necessary length for deployment of such instruments. Accordingly, a need exists in the art for instrument delivery devices that allow for robust performance of the instrument that is being delivered.

### SUMMARY OF THE INVENTION

Provided herein is a medical device, including an instrument having a proximal end and a distal end, an introducer configured to moveably receive the instrument and having an outer housing having a proximal end, a distal end, and a sidewall therebetween defining an inner volume, and an inner housing having a proximal end, a distal end, and a sidewall therebetween, the distal end of the introducer configured to couple the introducer to an intravenous line, wherein the inner housing and outer housing are displaceable relative to one another to move the instrument between a first position, in which the instrument is disposed within the outer housing, and a second position, in which the distal end of the instrument is disposed beyond the distal end of the outer housing such that at least a first portion of the instrument is disposed within the intravenous line when the introducer is coupled to the intravenous line, such that a length of the inner housing and the outer housing when the instrument is in the first position is greater than when the instrument is in the second position.

### BRIEF DESCRIPTION OF THE DRAWINGS

FIG. 1 is a top view of an instrument delivery device and system according to non-limiting embodiments described herein;

FIG. 2 is a perspective view of an instrument delivery device and system according to non-limiting embodiments described herein;

FIGS. 3A and 3B are perspective and cross-section views of an instrument delivery device and system according to non-limiting embodiments described herein; and

FIG. 4 is a perspective view of an instrument delivery device and system according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

U.S. patent application Ser. No. 17/570,554 is incorporated herein by reference in its entirety.

Provided herein are devices and systems for delivering instruments through indwelling catheters, such as peripheral intravenous catheters (PIVCs). While certain devices (e.g., blood draw devices) are discussed below in terms of devices that may be used with PIVCs, and exemplified in the attached drawings, those of skill will appreciate that any number of different devices for introducing an instrument, including instruments ranging from tubes, probes, sensors (e.g., pressure sensors, pH sensors, lactate sensors, glucose sensors, and the like), wiring, fiber optics, guidewires, etc., may be used within the scope of the present disclosure.

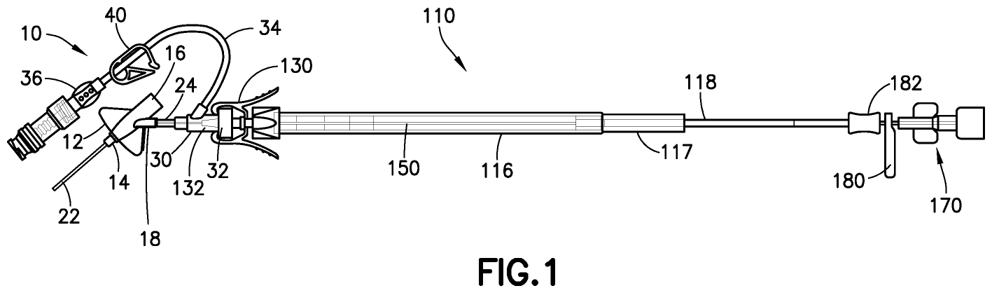

Referring now to FIG. 1, shown is a non-limiting embodiment of a system including a catheter assembly 10 and instrument delivery device 110. Suitable catheter assemblies for use with instrument delivery devices described herein are commercially available, for example from Becton, Dickinson and Company under the trade name Nexiva. Cather assembly 10 may include a catheter adapter 12, which may include a distal end 14 and a proximal end 16. In some embodiments, the catheter adapter 12 may include one or more additional ports 18. In some embodiments, port 18 may be disposed between the distal end 14 and the proximal end 16. In some embodiments, more than one port 18 may be disposed between the distal end 14 and the proximal end 16. In some embodiments, port 18 may be disposed at proximal end 16. In some embodiments, the first catheter adapter 12 may include a first lumen extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end and a proximal end, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, a 60, or a 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

Catheter assembly 10 may include a needleless access connector 32 and/or a second fluid conduit 34. Suitable needleless access connectors 32 can include any split-septum connector and/or those with direct fluid path access. Needleless access connectors 32 are known to those of skill in the art and are commercially available from, for example, Becton, Dickinson and Company under the trade names Q-SYTE, and SMARTSITE. While the non-limiting embodiments of FIG. 1 show a needleless access connector arranged at connector 30, those of skill in the art will appreciate that suitable needleless access connectors may also be arranged at luer 36. In non-limiting embodiments, needleless access connector 32 includes a septum (not shown), such as a self-healing septum. In non-limiting embodiments, the septum is a slit-type septum. As will be described below, instrument delivery device 110 may be reversibly coupleable to the needleless access connector 32, and one or more portions of the instrument delivery device may pierce the septum and access the patient's vasculature through catheter 22.

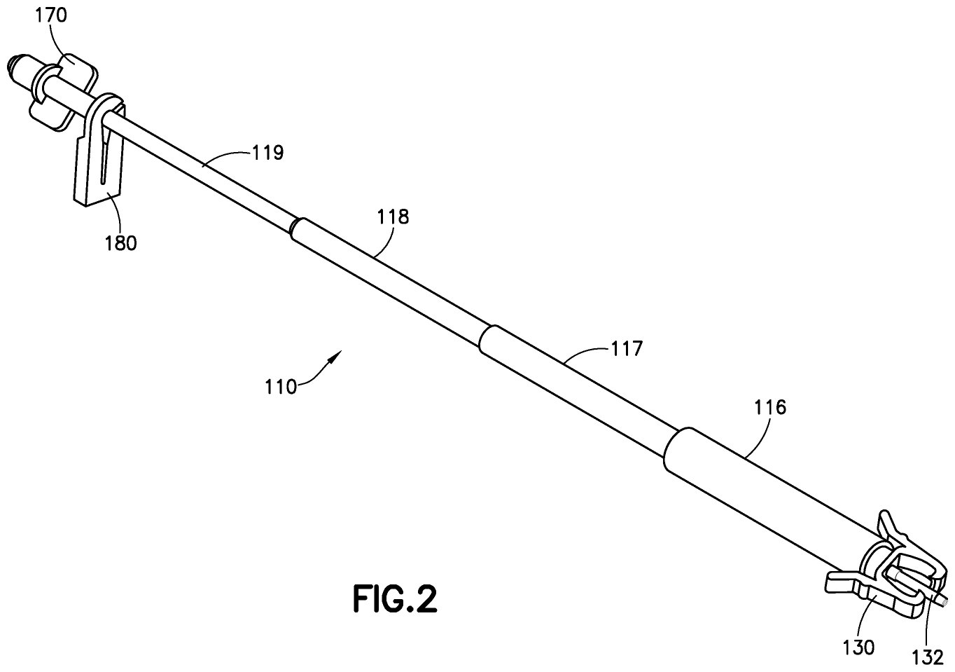

With continuing reference to FIGS. 1 and 2, instrument delivery device 110 includes an outer housing 116 having a proximal end and a distal end, and an inner housing. Throughout the illustrated embodiments, it will be clear to those of skill in the art that an intended effect of the instrument delivery devices described herein is that, when an instrument has been delivered (e.g., instrument has moved from a first configuration, where instrument does not extend beyond distal end of outer housing 116, to a second configuration, where instrument extends beyond distal end of outer housing 116), a total length of the housing(s) of instrument delivery device is less than when the instrument has not been delivered. By assuming a shorter length when the instrument has been delivered, errors associated with site manipulation can be reduced.

Inner housing, as shown in the non-limiting embodiment of FIGS. 1 and 2, may include a plurality of telescoping portions, for example telescoping portions 117, 118. In non-limiting embodiments, a first telescoping section 118 is slidably received within a second telescoping section 117. In non-limiting embodiments, second telescoping section 117 is slidably received within outer housing 116, such that inner housing may be slidably received entirely, or almost entirely, within outer housing 116. Each portion of inner housing includes a proximal end and a distal end and, in non-limiting embodiments, inner housing portions 117, 118 may have a variable diameter, to allow for the telescoping relationship. Instrument delivery device 110 further includes an instrument, exemplified in the drawings as catheter or fluid conduit 150 having a proximal end and a distal end 152, but, as described previously and as will be appreciated by those of skill in the art, may be any medical instrument that can be delivered through catheter assembly 10 to a patient's vasculature. Fluid conduit 150 is slidably received within outer housing 116, and may be advanced and/or retracted relative to outer housing 116 by displacement of inner housing relative to outer housing 116.

In non-limiting embodiments, fluid conduit 150 may be advanced from a first positon, in which distal end 152 of fluid conduit 150 is within instrument delivery device 110, for example within outer housing 116 and/or lock 130, and a second position, in which a distal end 152 of fluid conduit 150 is positioned distally of lock 130 and, in embodiments in which instrument delivery device 110 is coupled to catheter assembly 10, optionally distally of catheter 22. While lock 130 is exemplified as a proboscis 132 and arms in FIGS. 1 and 2, those of skill will appreciate that any type of suitable connection may be used to secure instrument delivery device 110 to an indwelling catheter, such as catheter assembly 10, including luer connections, clips, blunt plastic cannulae, blunt metal cannulae, hybrid luers (e.g., with a cannula) friction fits, and the like. Instrument delivery device 110 may further include, in some embodiments, a connector 170 arranged at proximal end of fluid conduit 150 to allow for various medical devices to be attached to inner housing 118, a clamp 180 for occluding flow through fluid conduit 150, and/or a grip 182 for aiding in displacing inner housing 118. Clamp 180 may be a slide clamp, for example as shown in FIG. 1, or may be a pinch clamp. Suitable connectors 170 include luer connectors, luer lock access devices, needless access connectors, and the like known to those of skill in the art.

With continuing reference to FIGS. 1 and 2, though not limited to those illustrated embodiments, in non-limiting embodiments, various portions 117, 118, and/or 119 of inner housing may include a lubricant therebetween, and/or between an inner housing portion and outer housing 116, and/or between the instrument and the inner 117, 118, and/or 119 and/or outer housing 116, so as to reduce the force needed to advance the instrument (such as fluid conduit 150).

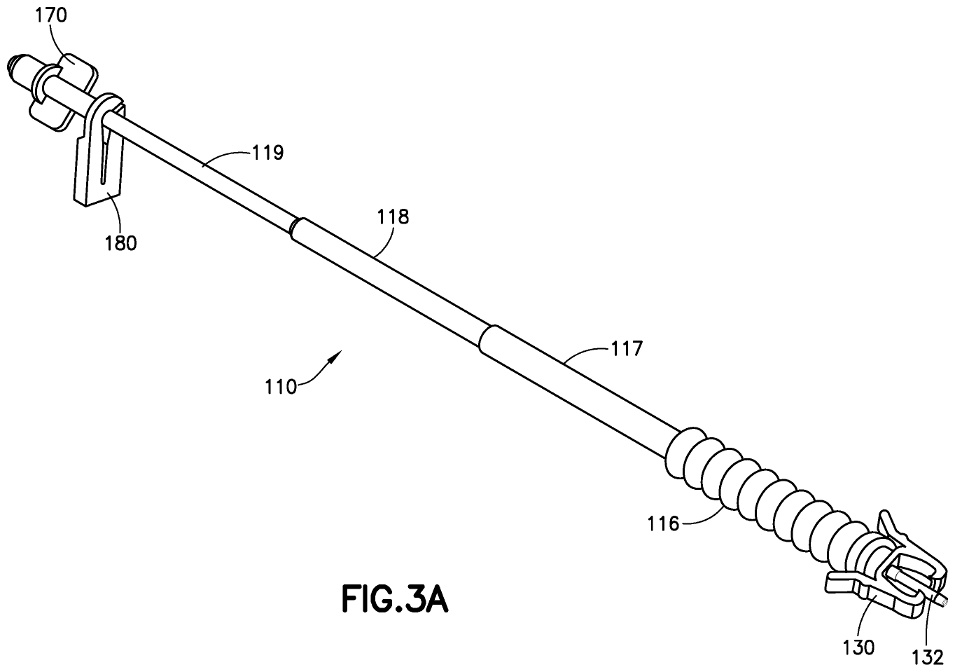

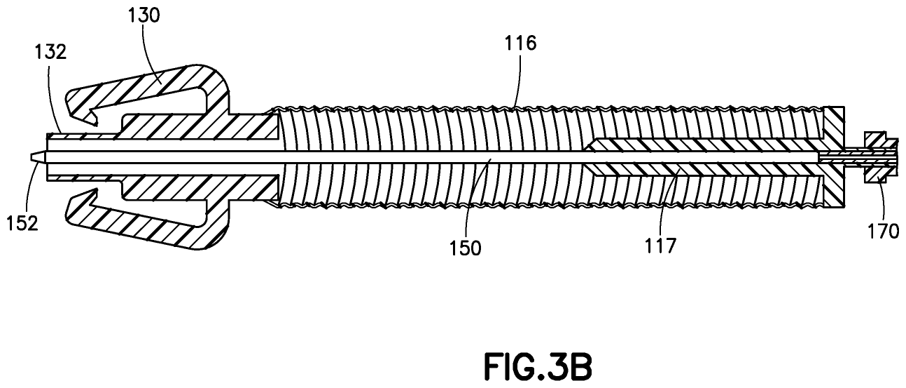

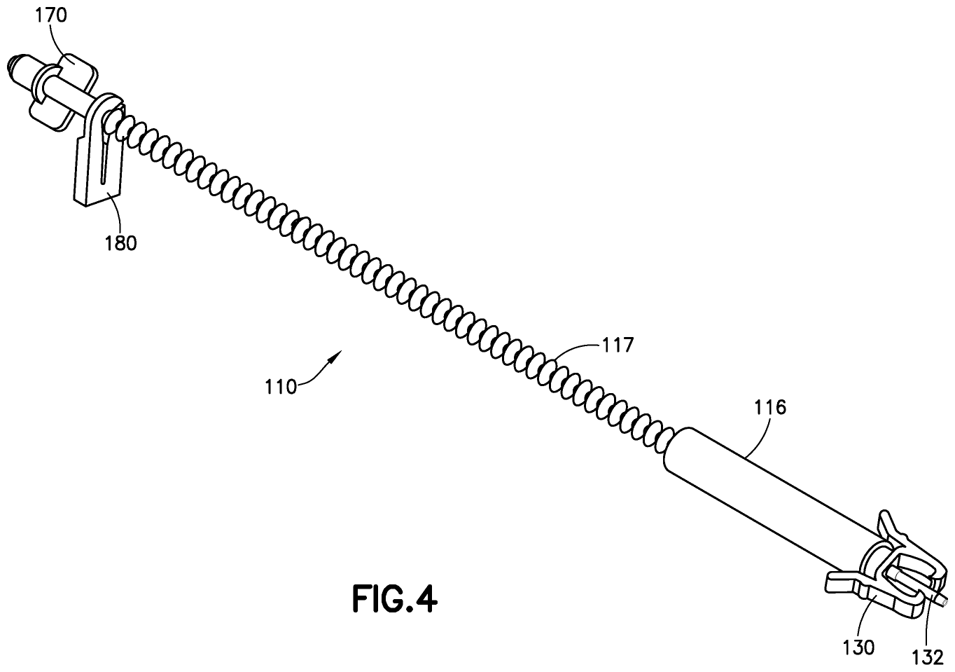

Turning to FIGS. 3A-4, shown are various embodiments of an instrument delivery device 110, in which outer housing 116 and/or inner housing 117 is collapsible. By “collapsible,” it is meant that the housing (or housing portion) is capable of collapsing along a longitudinal axis, from a first, greater length to a second, lesser length. Suitable collapsible materials, including plastics such as polyethylene, polypropylene, polyvinyl chloride, and the like, as well as elastomeric materials such as silicones, rubbers (natural and synthetic) and the like, and thermoplastic elastomers, and arrangements, such as an accordion arrangement, are known to those of skill in the art. With regard to FIGS. 3A and 3B, shown is an embodiment of instrument delivery device 110 in which outer housing 116 is collapsible and inner housing 117, 118, 119 is rigid. Outer housing 116 may be collapsible between a first, expanded configuration to a second, collapsed configuration, optionally during advancement of instrument (e.g., fluid conduit 150) by advancing inner housing 117, 118, 119 relative to outer housing 116. As shown in FIG. 3A, inner housing 117, 118, 119 may be telescoping, such that a process of advancing the instrument involves nesting a first housing portion 119 within a second housing portion 118, and nesting second housing portion 118 within third housing portion 117. During, or following, advancement of the instrument, outer housing 116 may collapse, reducing the overall effective length of instrument delivery device 110. While FIG. 3A shows a telescoping inner housing, those of skill will appreciate that inner housing 117, 118, 119 may include more or fewer telescoping sections, or may be a unitary housing.

Turning to FIG. 4, shown is an embodiment in which outer housing 116 is a rigid housing, and inner housing 117 is a collapsible housing. As described above, a housing may be collapsible between a first, expanded configuration to a second, collapsed configuration, optionally during advancement of instrument (e.g., fluid conduit 150) by advancing inner housing 117 relative to outer housing 116.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A medical device, comprising:
an instrument having a proximal end and a distal end;
an introducer configured to moveably receive the instrument and having:
an outer housing having a proximal end, a distal end, and a sidewall therebetween defining an inner volume; and
an inner housing having a proximal end, a distal end, and a sidewall therebetween,
the distal end of the introducer configured to couple the introducer to an intravenous line; and
wherein the inner housing and outer housing are displaceable relative to one another to move the instrument between a first position, in which the instrument is disposed within the outer housing, and a second position, in which the distal end of the instrument is disposed beyond the distal end of the outer housing such that at least a first portion of the instrument is disposed within the intravenous line when the introducer is coupled to the intravenous line, such that a length of the inner housing and the outer housing when the instrument is in the first position is greater than when the instrument is in the second position.

2. The medical device of claim 1, wherein the inner housing comprises a plurality of telescoping portions, at least one of the telescoping portions configured to be received within the inner volume of the outer housing.

3. The medical device of claim 2, wherein the inner housing comprises at least three telescoping portions, a first telescoping portion being slidably received within a second telescoping portion, and the second telescoping portion being slidably received within a third telescoping portion.

4. The medical device of claim 3, wherein the third telescoping portion is slidably received within the outer housing.

5. The medical device of claim 1, wherein the inner housing is rigid and the outer housing is collapsible longitudinally between a first configuration and a second configuration, wherein a length of the outer housing in the first configuration is greater than the length of the outer housing in the second configuration.

6. The medical device of claim 1, wherein the outer housing is rigid and the inner housing is collapsible longitudinally within the inner volume of the outer housing between a first configuration and a second configuration, wherein a length of the inner housing in the first configuration is greater than the length of the inner housing in the second configuration.

7. The medical device of claim 1, wherein the outer housing comprises a lock at the distal end thereof, the lock configured to couple the introducer to an intravenous line.

8. The medical device of claim 1, wherein the instrument is one or more of a catheter, guidewire, obturator, wire, electrical wiring, probe, light pipe, and sensor.

9. The medical device of claim 1, wherein the instrument is a catheter comprising a proximal end and a distal end.

10. The medical device of claim 9, further comprising a clamp arranged at the proximal end of the catheter, configured to selectively block fluid flow through the catheter.

11. The medical device of claim 9, wherein the catheter comprises a connecter at the proximal end thereof.

12. The medical device of claim 1, further comprising a lubricant arranged between the inner housing and the outer housing.

13. The medical device of claim 1, further comprising a lubricant between the instrument and the inner housing and/or the outer housing.

14. A system, comprising:
a catheter assembly comprising:
a catheter adapter, comprising:
a distal end;
a proximal end;
a lumen extending between the distal end and the proximal end; and
a side port arranged between the distal end and the proximal end, the side port in fluid communication with the lumen;
a catheter secured to the distal end of the catheter adapter and extending distally from the catheter adapter; and
a fluid conduit having a proximal end coupled to the side port and a distal end, the fluid conduit in fluid communication with the side port; and
the medical device of claim 1.
